(19) United States
(12) Patent Application Publication
(10) Pub. No.: US 2022/0412970 A1
(43) Pub. Date: Dec. 29, 2022

(54) Method and Apparatus for Processing Material

(71) Applicant: Life Technologies Holdings PTE LTD, Singapore (SG)

(72) Inventor: Jukka Tuunanen, Helsinki (FI)

(21) Appl. No.: 17/779,467

(22) PCT Filed: Dec. 1, 2020
(86) PCT No.: PCT/IB2020/061301

§ 371 Date: May 24, 2022

(30) Foreign Application Priority Data
Dec. 2, 2019 (FI) 20196049

(51) Int. Cl. G01N 33/543; B03C 1/033; B03C 1/28; B03C 1/30; G01N 35/00; G01N 1/40

(52) U.S. Cl. CPC G01N 33/54326; B03C 1/0332; B03C 1/288; B03C 1/30; G01N 35/0098; G01N 1/4077; B01L 2200/0668; B01L 2300/0832; B01L 2400/043; B03C 2201/18; B03C 2201/26; G01N 35/1065

(57) ABSTRACT

A target substance is collected from a composition by using magnetically responsive particles and a magnetic transfer probe. The composition may be prepared, e.g., by introducing magnetically responsive particles to a sample. The particles selectively bind to a target substance of the composition. The target substance and the particles are collected from the sample by using the magnetic transfer probe, which comprises a probe magnet. The probe magnet is a permanent magnet, which comprises a cylindrical portion and a convex bottom portion adjoining the cylindrical portion. The particle collection region of the magnetic transfer probe is at a low position, which allows collecting the particles from a small amount of the prepared composition.

### FIELD

Some variations relate to processing a composition by using magnetically responsive particles and by using a magnetic transfer probe.

### BACKGROUND

A composition may comprise a target substance and a liquid medium. The target substance may be separated from the liquid medium by using magnetically responsive particles. The particles may be arranged to selectively bind to the target substance. The particles may be collected and lifted from a vessel by using a magnetic transfer probe. The target substance bound to the particles may be collected and separated from the liquid medium together with the particles.

### SUMMARY

An object is to provide a method for processing a composition. An object is to provide a method for collecting a target substance. An object is to provide a method for transferring a target substance. An object is to provide a method for enriching a target substance. An object is to provide a method for purifying a target substance. An object is to provide an apparatus for processing a composition. An object is to provide an apparatus for collecting a target substance. An object is to provide an apparatus for transferring a target substance. An object is to provide an apparatus for enriching a target substance. An object is to provide an apparatus for purifying a target substance.

According to an aspect, there is provided a method for processing a composition (MX1) by using a magnetic transfer probe (100), the transfer probe (100) comprising a shield (120) and a probe magnet (MAG1) movable inside the shield (120),
the method comprising:
   providing a first composition (MX1) in a vessel (VES1), wherein the composition (MX1) comprises a first liquid (LIQ1) and a plurality of magnetically responsive particles (P1), wherein the particles (P1) are arranged to selectively interact with a target substance (M1),
   positioning the transfer probe (100) into the vessel (VES1) so as to collect the particles (P1) from the first composition (MX1),
   removing the collected particles (P1) together with the transfer probe (100) from the vessel (VES1) by causing a relative vertical movement between the transfer probe (100) and the vessel (VES1), and
   releasing the collected particles (P1) from the shield (120) to a release location (LOC2) by causing a relative vertical movement between the probe magnet (MAG1) and the shield (120),
   wherein the probe magnet (MAG1) is a permanent magnet, which comprises a cylindrical portion (SRF0) and a convex bottom portion (CNX1) adjoining the cylindrical portion (SRF0), the magnet has an axis of symmetry (AX1), the axis of symmetry (AX1) intersects the bottom portion (CNX1) at an intersection point (Q1), the intersection point (Q1) and the circular lower boundary (CIR2) of the cylindrical portion (SRF0) define a reference cone (REF0), and the bottom portion (CNX1) protrudes with respect to the reference cone (REF0).

The method comprises using the transfer probe to collect and/or process magnetically responsive particles of the composition. The transfer probe comprises a permanent probe magnet. The probe magnet comprises a cylindrical portion and a convex bottom portion. The probe magnet having the convex bottom may allow operation in a very small volume of the composition. The convex bottom portion of the magnet may be, e.g., a hemisphere or a truncated hemisphere.

A composition may comprise a liquid component and magnetically responsive particles. The composition may be contained in a vessel. The transfer probe may be used for collecting the magnetic particles from the composition contained in the vessel and/or releasing the magnetically responsive particles to a release location. The composition may further comprise a target substance. The magnetically responsive particles may selectively bind to the target substance, so as to selectively collect and/or process the target substance. The method may be used, e.g., in order to collect, enrich, purify and/or transfer the target substance.

The collected particles may be optionally analyzed, e.g., by an analytical instrument. The method may be used, e.g., in order to analyze whether a sample contains the target substance or not. The method may comprise measuring an amount and/or concentration of the target substance, after the target substance has been collected by using the magnetically responsive particles and the transfer probe.

The composition may be prepared, e.g., by introducing magnetically responsive particles to a sample. The magnetically responsive particles may selectively bind to the target substance of the composition. The target substance and the magnetically responsive particles may be collected from the sample simultaneously.

The transfer probe may collect magnetically responsive particles to a collection region. A maximum distance between the collection region and the lowermost point of the probe may be small, thanks to the convex bottom portion of the magnet. The distance between the collection region and the lowermost point of the probe may be small, thanks to the convex bottom portion of the magnet.

The convex bottom portion of the magnet may have a doubly curved surface portion, which may provide a high gradient of the magnetic field at the collection region of the transfer probe. The doubly curved surface portion may be, e.g., a substantially spherical surface portion. The magnetically responsive particles may be mainly attracted to the collection region where the gradient of the magnetic field of the probe has a maximum.

The magnetically responsive particles may be attracted by the magnetic field generated by the permanent magnet. The magnetic field may collect the particles to the collection region of the transfer probe. The magnitude of the magnetic field generated by the probe magnet may increase with increasing diameter of the probe magnet. The particles may be collected more effectively when using a probe magnet which has a large diameter. However, using a probe magnet which has a large diameter may make it more difficult to release the collected particles to a small volume of liquid. Thanks to the convex bottom portion, the collection region of the probe may be suitable for operation in a small volume, wherein the diameter of the probe magnet may be large enough for generating a sufficient magnetic field.

The vertical position of the collection region may be significantly below the cylindrical portion of the magnet. Using the probe magnet with the convex bottom portion may facilitate collecting the particles from a small volume of a liquid and/or may facilitate releasing the particles to a small volume of a liquid.

The convex shape of the bottom portion may provide a magnetic field, where the maximum gradient is located significantly below the cylindrical portion of the probe magnet. The collecting force which pulls the magnetically responsive particles towards the transfer probe may be substantially proportional to the magnitude of the gradient of the magnetic field. The transfer probe may collect the magnetically responsive particles mainly to the collecting region, which is located at the bottom portion of the transfer probe below the cylindrical portion of the probe magnet. The convex shape of the bottom portion may allow using the transfer probe in a reduced volume of a liquid, thanks to the low vertical position of the particle collecting region.

The transfer probe may be suitable for use in a small liquid volume. The magnetically responsive particles may be collected from a small liquid volume and/or the magnetically responsive particles may be released to a small liquid volume.

The particles may be collected from a first composition MX1, wherein the lower limit of the volume of the first composition MX1 may be, e.g., in the range of 5 μl to 50 μl.

The reduced volume of the liquid may allow analysis by using a reduced amount of a sample. The reduced volume of the liquid may allow distributing an amount of a sample to several sample wells. The reduced volume of the liquid may reduce consumption of the magnetically responsive particles. The reduced volume of the liquid may reduce consumption of reagents and/or reactants. The reduced volume may allow increasing processing speed. The reduced volume may allow increasing analysis speed. The reduced volume of the liquid may allow reducing the amount of waste.

The lower limit of the volume of a liquid at the release location may also be small. The lower limit of the volume of a liquid at the release location may be, e.g., in the range of 5 μl to 50 μl. The lower limit of the volume of the liquid at the release location may be, e.g., in the range of 5 μl to 15 μl, e.g., in order to provide an increased concentration of the collected particles P1 and/or to provide an increased concentration of a target substance M1.

The volume of the first composition MX1 may optionally by substantially greater than the volume of a liquid at the release location, e.g., in order to provide an increased enrichment ratio.

The transfer probe may be arranged to transfer magnetically responsive particles, e.g., in order to manufacture a product. The transfer probe may be arranged to transfer magnetically responsive particles, e.g., in order to purify a substance. The transfer probe may be arranged to transfer magnetically responsive particles, e.g., in order to analyze a sample. The target substance may be collected, e.g., in order to produce a medicament, or in order to produce a chemical substance for an assay.

### BRIEF DESCRIPTION OF THE DRAWINGS

In the following examples, several variations will be described in more detail with reference to the appended drawings, in which

FIG. 1a shows, by way of example, a composition, which comprises a first liquid and a target substance,

FIG. 1b shows, by way of example, a composition which comprises the first liquid, the target substance, and magnetically responsive particles,

FIG. 1c shows, by way of example, gathering the magnetically responsive particles by using a magnetic field,

FIG. 2 shows, by way of example, in a cross-sectional view, a probe magnet, a shield, and a vessel,

FIG. 3a shows, by way of example, in a cross-sectional view, a transfer probe and an amount of the composition,

FIG. 3b shows, by way of example, in a cross-sectional view, collecting particles to the transfer probe,

FIG. 3c shows, by way of example, in a cross-sectional view, separating the particles from the first liquid,

FIG. 3d shows, by way of example, in a cross-sectional view, particles attached to the probe,

FIG. 4a shows, by way of example, in a cross-sectional view, the probe and a second liquid,

FIG. 4b shows, by way of example, in a cross-sectional view, immersing the probe with the particles into the second liquid,

FIG. 4c shows, by way of example, in a cross-sectional view, releasing the particles by lifting the magnet with respect to the shield,

FIG. 4d shows, by way of example, in a cross-sectional view, lifting the probe from the second liquid,

FIG. 5a shows, by way of example, in a cross-sectional view, the position of the collecting region provided by the probe magnet and the position of the collecting region provided by a comparative magnet,

FIG. 5b shows, by way of example, in a cross-sectional view, the position of the collecting region provided by the probe magnet and the position of the collecting region provided by a comparative magnet,

FIG. 6a shows, by way of example, in a cross-sectional view, the magnetic field generated by the probe magnet,

FIG. 6b shows, by way of example, in a cross-sectional view, the dimensions of a gap between the probe and a vessel,

FIG. 7a shows, by way of example, in a three-dimensional view, a probe magnet which has a convex bottom portion,

FIG. 7b shows, by way of example, in a cross-sectional view, a probe magnet which has a convex bottom portion,

FIG. 8a shows, by way of example, in a cross-sectional view, the spatial distribution of the magnetic field generated by a probe magnet,

FIG. 8b shows, by way of example, in a cross-sectional view, the spatial distribution of the magnetic field generated by a probe magnet,

FIG. 8c shows, by way of example, in a cross-sectional view, the spatial distribution of a magnetic field generated by a reference magnet, which has a flat end,

FIG. 9a shows, by way of example, in a cross-sectional view, a convex bottom portion which is a half of a spheroid,

FIG. 9b shows, by way of example, in a cross-sectional view, a convex bottom portion which is a truncated hemisphere,

FIG. 9c shows, by way of example, in a cross-sectional view, a convex bottom portion which has a combination of conical surfaces,

FIG. 10 shows, by way of example, in a cross-sectional view, an apparatus, which comprises the transfer probe,

FIGS. 11a to 11d show releasing the transferred particles to a substantially planar surface,

FIGS. 12a and 12b show, by way of example, in a cross-sectional view, a transfer probe which has substantially spherical bottom,

FIG. 12c shows, by way of example, in a cross-sectional view, the shape of the bottom of the vessel of FIGS. 12a and 12b,

FIG. 12d shows, by way of example, in a cross-sectional view, using the vessel of FIG. 12a together with a transfer probe which has a tip,

FIG. 12e shows, by way of example, in a cross-sectional view, a transfer probe,

FIG. 12f shows, by way of example, in a cross-sectional view, the transfer probe of FIG. 12f positioned in a vessel,

FIG. 12g shows, by way of example, in a cross-sectional view, an array of transfer probes, and an array of wells, and

FIG. 12h shows, by way of example, in a cross-sectional view, an array of transfer probes, and an array of wells.

### DETAILED DESCRIPTION

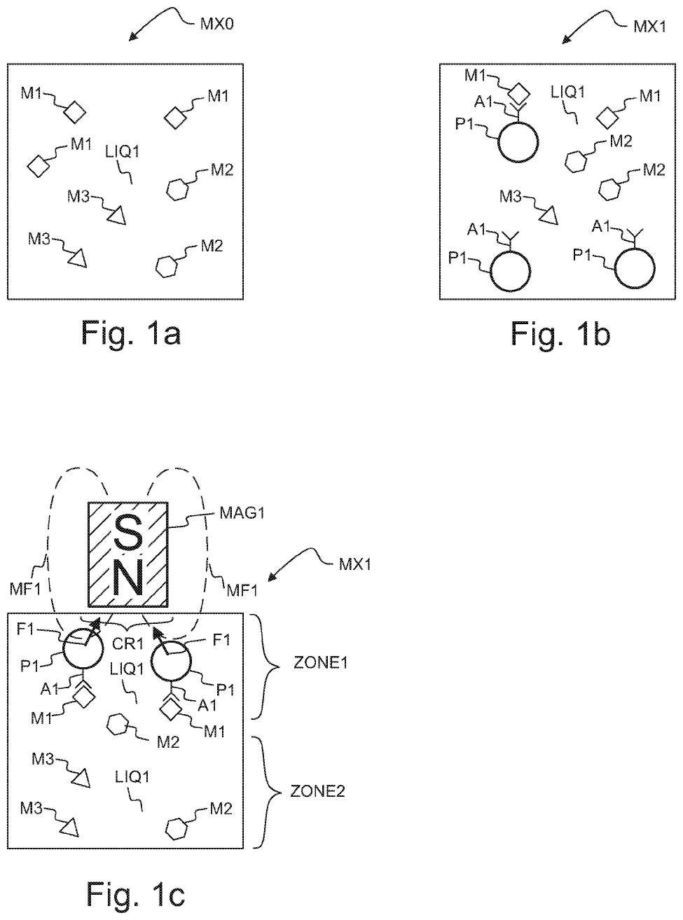

Referring to FIG. 1a, a primary composition MX0 may comprise one or more substances M1, M2, M3 and a liquid medium LIQ1. The composition MX0 may be, e.g., a sample which comprises one or more substances M1, M2, M3 and a liquid LIQ1. The composition MX0 may be a mixture which comprises one or more substances M1, M2, M3 and a liquid LIQ1. The composition MX0 may comprise a target substance M1.

The composition MX0 may be, e.g., a biological sample. The target substance M1 may, e.g., consist of cells (e.g., bacteria or cancer cells), proteins (e.g., antigens or antibodies), enzymes, or nucleic acids.

Referring to FIG. 1b, a composition MX1 may comprise a plurality of magnetically responsive particles P1, one or more substances M1, M2, M3 and a liquid medium LIQ1. The composition MX1 may be obtained, e.g., by introducing magnetically responsive particles P1 to the primary composition MX0. The composition MX1 may be a mixture which comprises magnetically responsive particles P1, one or more substances M1, M2, M3 and a liquid medium LIQ1. Magnetically responsive particles P1 may be added to a sample MX0 so as to form a suspension MX1, which comprises the particles P1 suspended in the liquid LIQ1.

The particles P1 may selectively interact with the target substance M1. The particles P1 may be arranged to selectively bind to the target substance M1 of the sample MX0 but not to a second substance M2 of the sample MX0. The magnetically responsive particles P1 may comprise binding sites Al to selectively bind to the target substance M1. The particles P1 may be selectively bound to the target substance M1, but not to the substances M2, M3. The magnetically responsive particles P1 may also be called, e.g., as magnetic beads.

The magnetically responsive particles P1 may be used for separating a specific target substance M1 from a liquid medium LIQ1. The particles P1 may be coated, e.g., with a specific reagent Al, which may selectively interact with the target substance M1. The particles P1 may be coated, e.g., with an affinity reagent for the target substance M1. The material of the particles P1 may also be selected to intrinsically interact with the target substance M1. For example, a silica surface may interact with nucleic acids even without an additional coating.

The size of the magnetically responsive particles P1 may be, e.g., in the range of 50 nm to 10 μm. The size of the magnetically responsive particles may be, e.g., in the range of 0.5 μm to 5 μm. The size of the magnetically responsive particles may be, e.g., substantially equal to 1 μm or 2.8 μm. The size of the magnetically responsive particles may be, e.g., substantially equal to 3 μm. The material of the magnetically responsive particles P1 may be selected such that the particles P1 may be attracted to a magnet MAG1. The magnetically responsive particles P1 may be, e.g., ferromagnetic particles, ferrimagnetic particles, or superparamagnetic particles. The material of the magnetically responsive particles P1 may be selected such that the particles P1 are not permanent magnets, wherein the magnetically responsive particles P1 may be magnetizable. A large variety of such particles P1 are commercially available.

Referring to FIG. 1c, the magnetically responsive particles P1 and the target substance M1 bound to the particles P1 may be collected from the composition MX1 by using a magnetic field MF1 generated by a magnet MAG1. The particles P1 may be collected from the composition by using a magnetic field MF1 generated by a permanent magnet MAG1. The magnetic field MF1 may move the particles P1 towards a collection region CR1 of a surface SRF1. A major part of the particles P1 may be collected to the collection region CR1 in the vicinity of the magnet MAG1. Substantially all particles P1 may be eventually collected to the collection region CR1.

Collecting the particles P1 may modify the composition MX1 such that the composition MX1 has an enriched zone ZONE1 and a depleted zone ZONE2. The concentration of the particles P1 in the enriched zone ZONE1 may be substantially higher than the concentration of the particles P1 in the depleted zone ZONE2. The concentration of the particles P1 in the depleted zone ZONE2 may be, e.g., substantially equal to zero. The particles P1 may selectively bind to the target substance M1 such that the concentration of the target substance M1 in the enriched zone ZONE1 may be substantially higher than the concentration of the target substance M1 in the depleted zone ZONE2.

A magnetically responsive particle P1 may be moved by a force F1, which is substantially proportional to the gradient of the magnetic field MF1. The magnetically responsive particles P1 may be mainly attracted to a region CR1 of a surface where the gradient of the magnetic field MF1 attains a maximum value. The region of the maximum gradient may operate as a collection region for the particles P1.

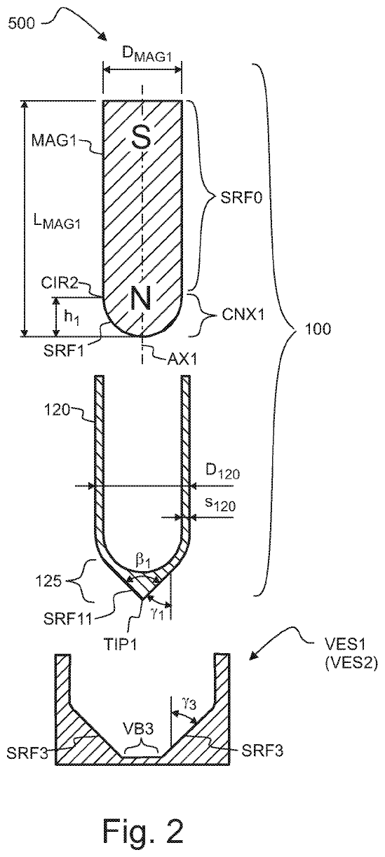

Referring to FIG. 2, an apparatus 500 may comprise a transfer probe 100 and a vessel VES1 and/or VES2. The transfer probe 100 may comprise a shield 120 and a permanent magnet MAG1 movable within the shield 120. The magnet MAG1 may be an elongated rod, which may be moved up and down with respect to the shield 120. The probe magnet MAG1 may be moved up and down within the hollow shield 120. The shield 120 may be hollow and the shield may have a closed bottom portion 125. The bottom portion 125 may be, e.g., a tapered portion. The bottom portion 125 may be, e.g., a tapered portion with a tip TIP1.

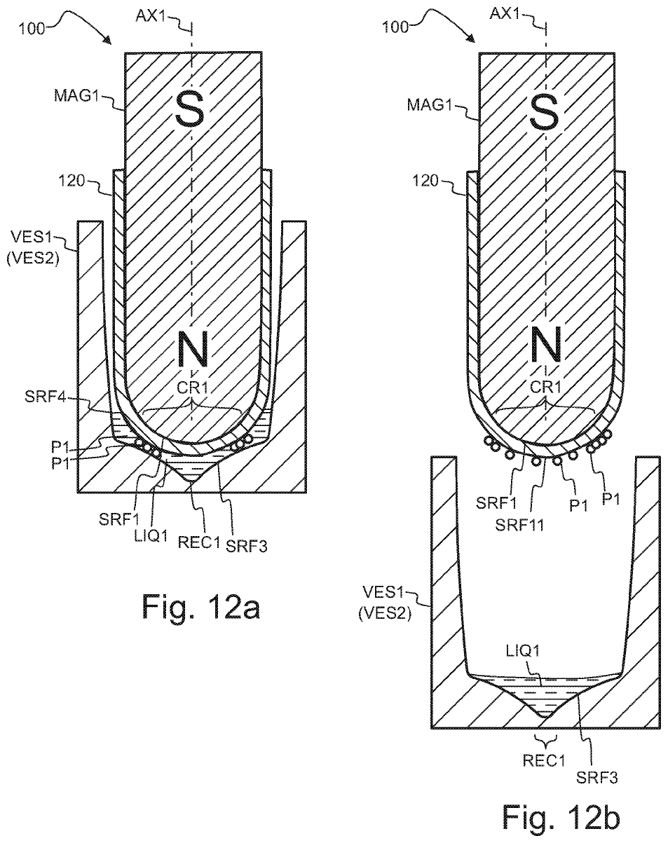

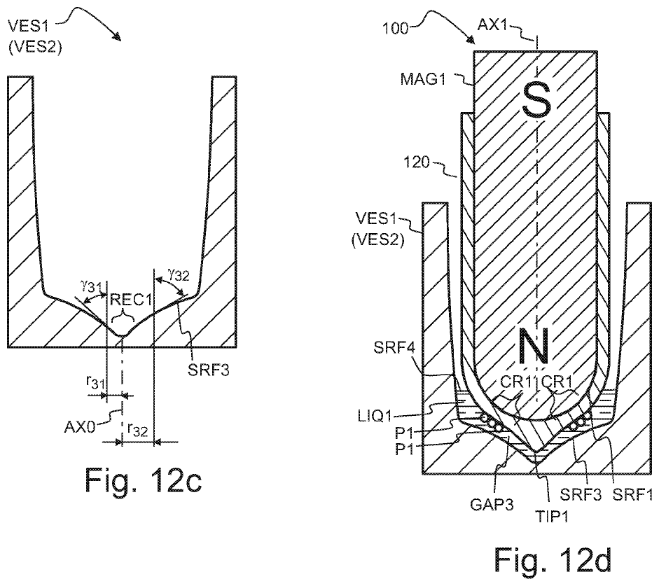

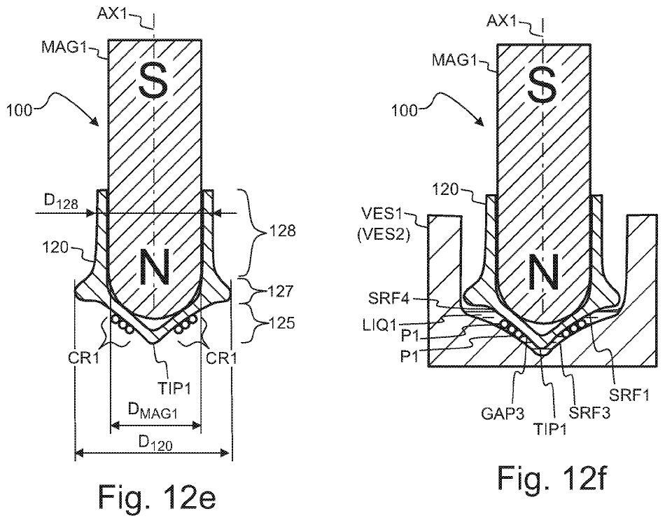

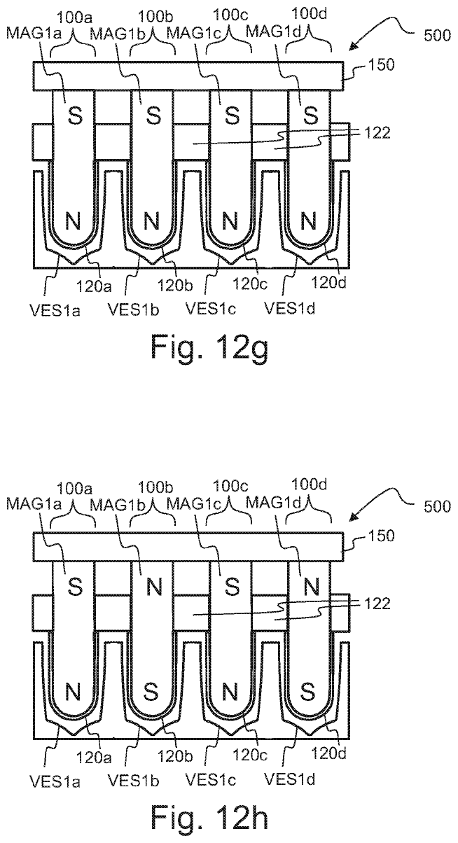

The bottom portion of the shield 120 may have an outer surface SRF11. The outer surface SRF11 may comprise, e.g., a tapered bottom portion 125 (FIG. 2, FIG. 12d, FIG. 12e). The outer surface (SRF11) of the shield 120 and the inner bottom surface (SRF3) of the vessel VES1, VES2 may be substantially axially symmetric.

The bottom portion 125 may also be, e.g., a substantially spherical portion (FIG. 12a). The outer surface SRF11 may have, e.g., a substantially spherical form (FIG. 12a).

The magnet MAG1 may comprise a cylindrical portion SRF0 and a convex bottom portion CNX1 adjoining the cylindrical portion SRF0. The convex bottom portion CNX1 may consist of the same permanently magnetic material as the cylindrical portion SRF0. The convex bottom portion CNX1 and the adjoining the cylindrical portion SRF0 may together form, e.g., a single body.

The probe magnet MAG1 may have a diameter D_{MAG1} and a length L_{MAG1}. The probe magnet MAG1 may have a substantially cylindrical surface portion SRF0 and a convex bottom portion CNX1 adjoining the cylindrical portion SRF0. The bottom portion CNX1 may have a height h₁. The cylindrical portion SRF0 may have a circular lower boundary CIR2. The symbol SRF1 denotes the surface of the bottom portion CNX1. The probe magnet MAG1 may be axially symmetric with respect to a vertical symmetry axis AX1.

The diameter D_{MAG1} of the probe magnet MAG1 may be, e.g., in the range of 1 mm to 8 mm, advantageously in the range of 3 to 5 mm. The diameter D_{MAG1} of the probe magnet MAG1 may be, e.g., substantially equal to 1.6 mm, 3 mm, 4 mm or 7.6 mm. The probe magnet MAG1 may have, e.g., a hemispherical bottom portion CNX1. The height h₁ of the bottom portion CNX1 may be, e.g., in the range of 40% to 60% of the diameter D_{MAG1}.

The magnet MAG1 may be so long that the upper pole of the magnet MAG1 is kept above the surface of the liquid LIQ1. The ratio of the length L_{MAG1} to the diameter D_{MAG1}. may be, e.g., greater than or equal to 2.0, advantageously greater than or equal to 4.0. The thickness 5120 of the wall of the shield 120 may be, e.g., in the range of 1% to 20% of the diameter D_{MAG1}. The thickness 5120 of the wall of the shield 120 may be, e.g., in the range of 0.3 mm to 0.5 mm. The shield 120 may have an outer diameter D₁₂₀.

The material of the shield 120 may be selected such that the shield 120 does not modify the magnetic field of the magnet MAG1. The relative magnetic permeability of the material of the shield 120 may be substantially equal to one. The material of the shield 120 may be, e.g., polymer or glass. The material of the shield 120 may be, e.g., polypropylene, polyethylene or polycarbonate.

The bottom portion 125 of the shield 120 may optionally have, e.g., a tapered surface SRF11 with a tip TIP1. The tapered portion 125 of the surface SRF11 may have an apex angle β₁, and a taper angle γ₁.

The apex angle β₁ of the tapered portion 125 of the shield 120 may be, e.g., in the range of 80° to 100°, advantageously in the range of 85° to 95°, and preferably substantially equal to 90°.

The taper angle γ₁ at the collecting region CR1 may be, e.g., in the range of 40° to 50°, e.g., when used together with a hemispherical bottom portion CNX1, e.g., in order to facilitate operation in a small liquid volume. The taper angle γ₁ of the surface SRF11 at the collecting region CR1 may be, e.g., in the range of 40° to 50°, e.g., when used together with a substantially hemispherical bottom portion CNX1. A tapered bottom portion 125 may provide, e.g., an annular collecting region CR1. The particles P1 may be attached, e.g., as a concentrated ring on the annular collecting region CR1.

The method may comprise collecting the particles P1 from a first vessel VES1 and/or releasing the particles P1 to a second vessel VES2. The vessel VES1 and/or the vessel VES2 may have an inner (bottom) surface SRF3.

The shape of the inner surface SRF3 of the vessel VES1 and/or VES2 may, e.g., substantially correspond to the shape of the outer surface SRF11 of the shield 120.

The inner surface SRF3 of the vessel may have, e.g., a tapered portion. The tapered portion may have a taper angle γ₃. The taper angle γ3 of the vessel may be selected to substantially correspond to the taper angle γ₁ of the shield. For example, the taper angle γ₃ may be, e.g., in the range of γ₁ to γ₁+5°.

The material of the vessel VES1 may be selected such that it does not modify the magnetic field of the magnet MAG1. The relative permeability of the material of the vessel VES1, VES2 may be substantially equal to one. The material of the vessel VES1 and/or VES2 may be, e.g., a polymer, e.g., polypropylene, polyethylene, or polycarbonate.

The vessel VES1, VES2 may optionally have a central portion VB3, which may adjoin a tapered portion of the vessel. The vessel VES1, VES2 may optionally have a central recessed portion REC1, which may adjoin a tapered portion of the vessel (FIG. 12c).

The vessel VES1, VES2 may be, e.g., sample well. The vessel VES1,VES2 may be, e.g., a well of a microwell plate. The microwell plate may also be called, e.g., as a micro-titration plate or a sample plate or a well plate. The microwell plate may also be called, e.g., as a micro-titration plate. The vessel VES1,VES2 may be, e.g., a well of a microwell plate. A well plate may comprise an array of wells. A well plate may comprise, e.g., 24, 96, or 384 wells.

In an embodiment, particles may be simultaneously lifted from several wells of a microwell plate, by using an array of the transfer probes 100.

The magnet MAG1 have, e.g., a hemispherical bottom portion CNX1. The minimum volume of the liquid in the vessel VES1 or VES2 may be, e.g., in the range of 5 μl to 20 μl. (1 μl=0.000001 liters=10⁻¹² m³).

The magnet MAG1 may be a single piece or a combination of several permanent magnets. The symbols S and N refer to the poles of the magnet MAG1. The north pole (N) of the magnet MAG1 may be above or below the south pole (S).

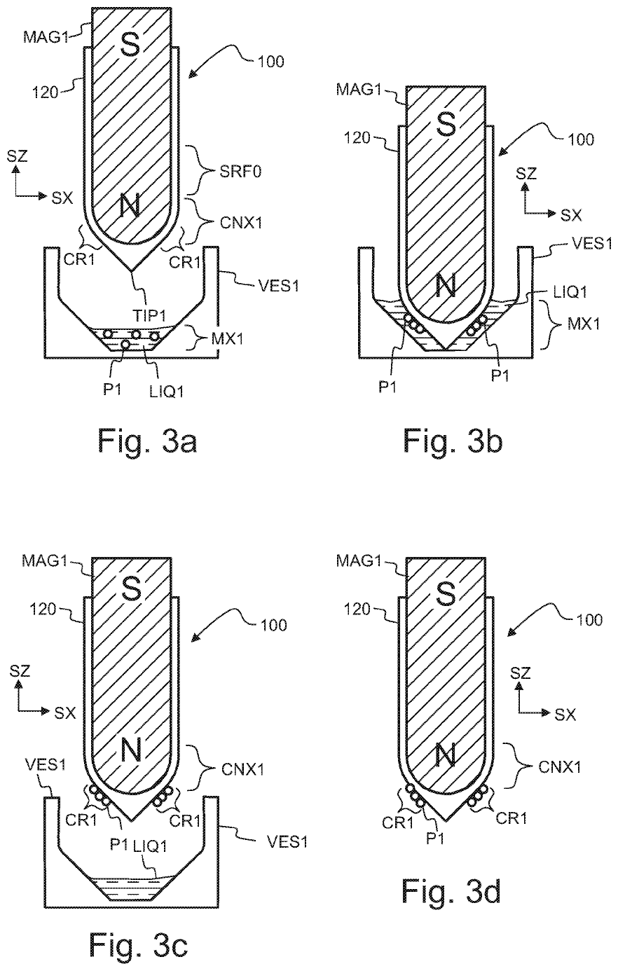

Referring to FIG. 3a, a vessel VES1 may contain an amount of the composition MX1. The composition MX1 may be obtained, e.g., by introducing the magnetically responsive particles P1 to a sample MX0. The magnet MAG1 may be moved to a lowermost position with respect to the shield 120. Moving the permanent magnet MAG1 to the lower position with respect to the shield 120 may effectively enable the magnetic field of the collecting region CR1. Moving the permanent magnet MAG1 to the lower position with respect to the shield 120 may move the maximum of the gradient of the magnetic field of the magnet MAG1 to a position, where the particles P1 are effectively attracted to the collecting region CR1.

The collecting region CR1 may be located, e.g., at a tapered portion of the shield 120. The collecting region CR1 may be located, e.g., at a spherical portion of the shield 120 (FIG. 12a).

Referring to FIG. 3b, the lower end of the transfer probe 100 may be inserted into the vessel VES1. The lower end of the probe 100 may immersed in the composition MX1 in order to collect the particles P1. The magnetic field of the probe may attract the particles P1 of the composition MX1 mainly to the collecting region CR1 of the probe 100. The collected particles P1 may, e.g., encircle the collecting region CR1 as an annular deposit of material. The particles P1 may be attached, e.g., as a concentrated ring on the collecting region CR1. An annular particle collection region CR1 may surround the bottom end of the shield 120 of the probe 100. The convex bottom portion CNX1 of the magnet MAG1 may provide an annular particle collection region CR1, which is located below the cylindrical portion SRF0 of the magnet MAG1.

The magnetic field of the probe 100 may convert the composition MX1 into a modified composition, which has an enriched zone and a depleted zone. The conversion may take place rapidly. Substantially all particles P1 may be eventually collected to the collecting region CR1.

Referring to FIGS. 3c and 3d, the magnetically responsive particles P1 may be lifted away from the liquid LIQ1 of the composition MX1 by lifting the probe 100. After the particles P1 have been collected to the probe 100, the probe 100 may be lifted out of the vessel VES1 keeping the magnet MAG1 still in its lower position, whereby the particles P1 may keep reliably attached to the probe 100. The probe magnet MAG1 may be at the lower position during the lifting so as to keep the particles P1 firmly attached to the collecting region CR1. The particles P1 may be lifted together with the probe 100. The particles P1 may be separated from the liquid LIQ1 by lifting the probe 100. Target substance M1 bound to the particles P1 may be substantially separated from the liquid LIQ1 by lifting the probe 100, in a situation where the composition MX1 contained the target substance M1. Target substance M1 bound to the particles P1 may be lifted from the vessel VES1. The probe 100 may be lifted by moving the probe 100 moved upwards and/or by moving the vessel VES1 downwards. The method may comprise causing a relative vertical movement between the probe 100 so as to remove the collected particles P1 together with the probe 100 from the vessel. Causing the vertical movement may comprise moving the probe upwards and/or moving the vessel downwards.

After removal from the vessel VES1, the particles P1 may be optionally washed, e.g., by temporarily immersing the probe 100 to a washing liquid. The washing may be carried out, e.g., so that the end of the probe 100 is placed in a washing liquid and the magnet MAG1 may be lifted, whereby the particles P1 may be released into the washing liquid. After washing, the particles P1 may be again collected by the probe 100 or by a different probe 100.

A small amount of the liquid LIQ1 may remain attached to particles P1 and/or to the probe 100 even after the probe 100 has been lifted from the vessel VES1. The volume of the liquid LIQ1 attached the particles P1 may be smaller than 1 μl. The attached liquid LIQ1 may be optionally evaporated away, if needed.

When the particles P1 are collected, the magnet MAG1 may be kept in its lower position, whereby the particles P1 may attach to the lower end of the shield 120. When the particles P1 are released, the magnet MAG1 may be lifted to its upper position, in which the magnet MAG1 no longer holds the particles P1 attached on the shield 120.

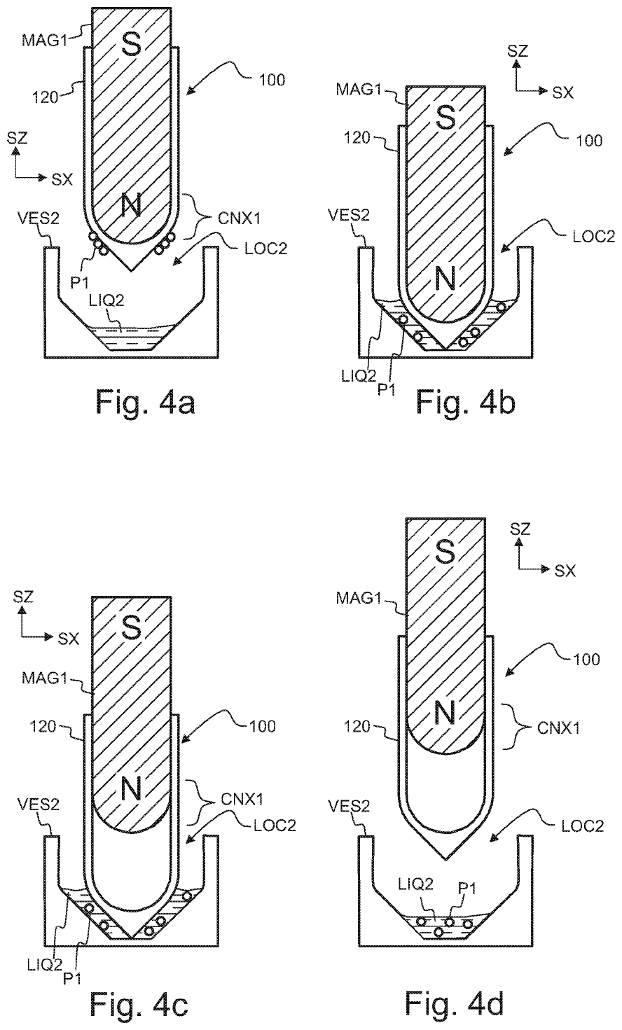

Referring to FIGS. 4a to 4d, magnetically responsive particles P1 temporarily attached to the probe 100 may be transferred and released to a release location LOC2. The bottom end of the probe 100 may be positioned to the release location LOC2. The probe 100 may be moved and contacted with a release surface and/or vessel at the release location LOC2. The magnet MAG1 of the probe 100 may be subsequently moved upwards with respect to the shield 120 of the probe 100 in order to temporarily reduce the magnitude of the magnetic field at the collecting region CR1 of the probe 100. Lifting the magnet MAG1 upwards with respect to the shield 120 may effectively disable the magnetic field of the collecting region CR1. The method may comprise causing a relative vertical movement between the magnet MAG1 and the shield 120, so as to release the collected particles P1 from the shield 120. Causing the vertical movement may comprise moving the magnet upwards and/or moving the shield downwards.

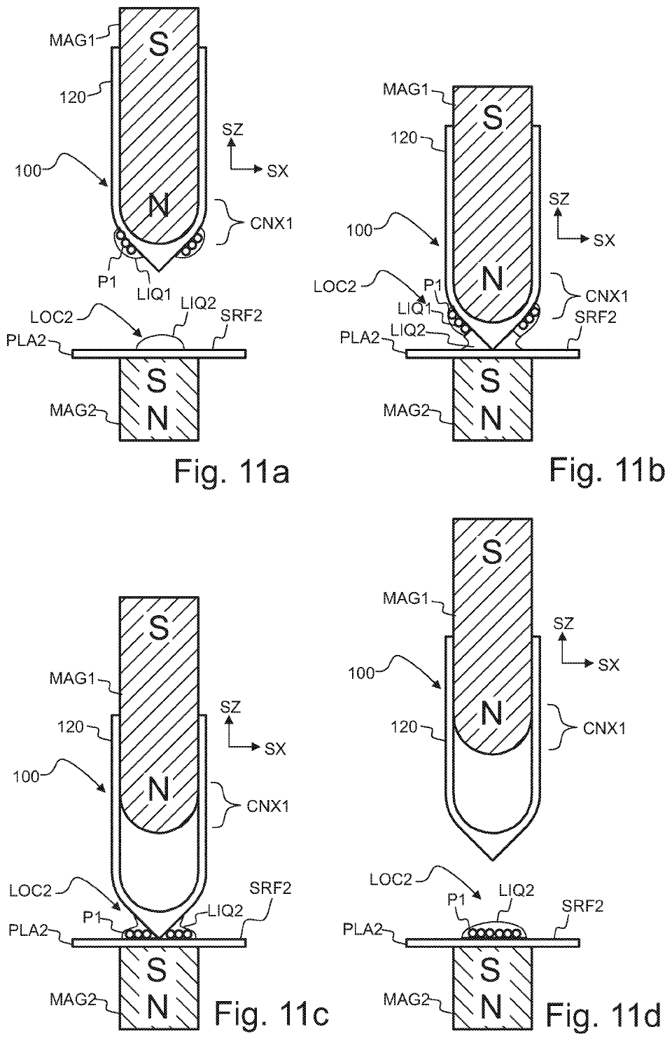

A second vessel VES2 or a sample plate PLA2 (FIG. 11a) may be arranged to operate as the release location LOC2. The (second) vessel VES2 may contain an amount of a (second) liquid LIQ2. The method may comprise releasing the particles P1 from the probe 100 to a liquid LIQ2 of a vessel VES2.

The liquid LIQ2 may facilitate release of the particles P1 from the probe 100 and/or the liquid LIQ2 may provide a suitable chemical environment for the target substance M1 carried by the particles P1. The surface tension of the liquid LIQ2 may facilitate release of the particles P1 from the probe 100, when the particles are brought in contact with the liquid LIQ2.

Release of the particles P1 may be optionally facilitated, e.g., by using an auxiliary release magnet MAG2, which may be located below the release location LOC2 (FIGS. 11a to 11d). However, the low vertical position of the collection region CR1 may allow releasing the particles P1 to the liquid LIQ2 also without using an auxiliary release magnet MAG2.

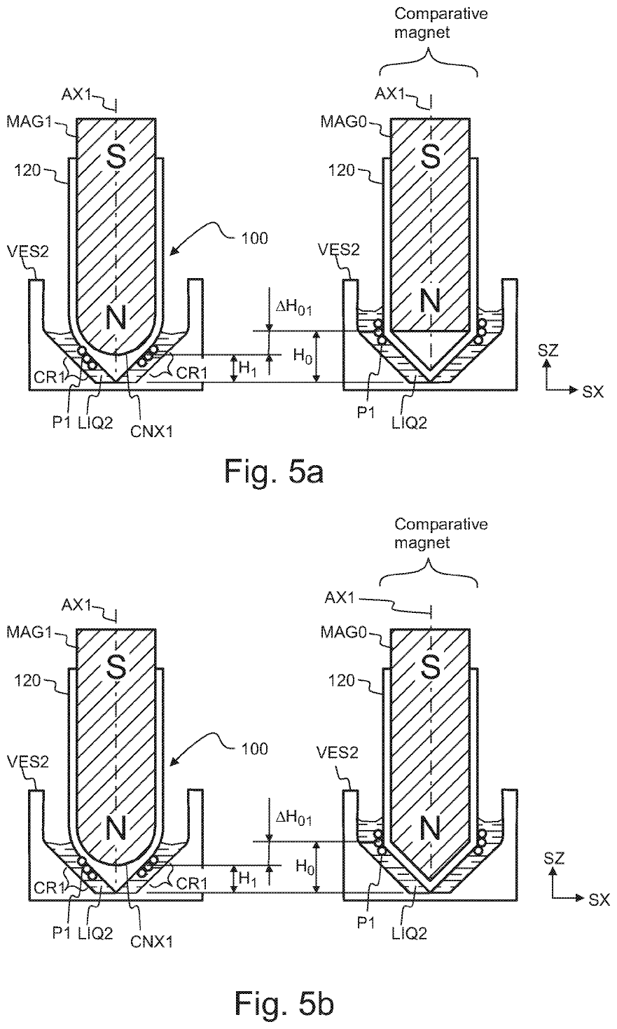

FIG. 5a and FIG. 5b illustrate the effect of the convex bottom portion CNX1 on the vertical position of the collection region CR1, when compared with a reference magnet MAG0.

Referring to FIG. 5a, the convex bottom portion CNX1 of the magnet MAG1 may provide a lower vertical position H¹ of the collecting region CR1, when compared with the vertical position H⁰ of a collecting region of a comparative cylindrical magnet MAG0, which has a planar end. The low position may allow reducing the volume of the liquid LIQ2 contained in the vessel VES2.

H₁ may denote the vertical position of the collecting region CR1 provided by the convex bottom portion CNX1, with respect to the bottom of the vessel VES2. H⁰ may denote the vertical position of a collecting region provided by reference magnet MAG0, with respect to the bottom of the vessel. ΔH₀₁ may denote the difference H₀−H₁. The relative difference ΔH₀₁/H₀ may depend on the shape of the convex bottom portion CNX1. The relative difference ΔH₀₁/H₀ may be, e.g., in the range of 10% to 60%. The shape of the convex bottom portion CNX1 may be selected such that the relative difference ΔH₀₁/H₀ is, e.g., in the range of 30% to 60%.

Table 1 shows, by way of example, a minimum volume V_{2,MIN} of liquid LIQ2 in the second vessel VES2 when using a probe magnet MAG1, which has hemispherical convex portion CNX1. The minimum volume V_{2,MIN} is shown for magnet diameters 1.6 mm, 3 mm, 4 mm and 7.6 mm.

TABLE 1
Examples for minimum and maximum volumes for magnet
diameters 1.6 mm, 3 mm, 4 mm, and 7.6 mm.

| | | | | Maximum |
|---|---|---|---|---|
| | | Minimum | | volume V_{1, MAX} |
| | Minimum | volume V_{2, MIN} | Maximum | of the |
| | volume V_{2, MIN} | of liquid LIQ2 | volume V_{2, MAX} | composition |
| | of liquid LIQ2 | in the second | of liquid LIQ2 | MX1 in the |
| | in the second | vessel VES2 | in the second | first vessel |
| | vessel VES2 | when using a | vessel VES2 | VES1 |
| | when using | reference | when using | when using |
| | the magnet | magnet MAG0 | the magnet | the magnet |
| | MAG1 with | with flat bottom | MAG1 with | MAG1 with |
| | hemispherical | end | hemispherical | hemispherical |
| Magnet | end portion | (Comparative | end portion | end portion |
| diameter | CNX1 | example) | CNX1 | CNX1 |
| 1.6 mm | 10 μl | 20 μl | 50 μl | 250 μl |
| 3.0 mm | 15 μl | 30 μl | 100 μl | 1000 μl |
| 4.0 mm | 20 μl | 50 μl | 150 μl | 1000 μl |
| 7.8 mm | 50 μl | 100 μl | 250 μl | 5000 μl |

Table 1 also shows, by way of comparison, minimum volume of liquid LIQ2 in the second vessel VES2 for different diameters of a reference probe magnet MAG0, which has a flat bottom. It may be noticed based on Table 1 that the magnet with the hemispherical bottom portion may allow releasing the particles P1 to a substantially smaller volume of the liquid LIQ2, when compared with the reference magnet of the same diameter. Table 1 also shows, by way of example, a maximum volume of the composition MX1 in the first vessel VES1. The volume of the liquid LIQ2 in the second vessel VES2 may be e.g., substantially smaller than the volume of the composition MX1 in the first vessel VES1, e.g., in order to enrich a substance (M1) from the composition MX1. A minimum volume of the composition MX1 in the first vessel VES1 may be, e.g., greater than or equal to the minimum volume of the liquid LIQ2 in the second vessel VES2, e.g., in order to enrich a substance (M1) from the composition MX1. Table 1 also shows, by way of example, a maximum volume of liquid LIQ2 in the second vessel VES2.

Referring to FIG. 5b, the convex bottom portion CNX1 of the magnet MAG1 may provide a lowered position H₁ of the collecting region CR1 also when compared with the vertical position H₀ of a collecting region of a comparative magnet MAG0, which has a conical bottom end with a sharp tip.

When using the comparative magnet MAG0 of FIG. 5a or 5b, the magnetically responsive particles P1 are typically attracted to an annular collecting region, which is located at the bottom end of the cylindrical portion of the comparative magnet MAG0. The magnetically responsive particles P1 are typically not attached to the sharp tip of conical portion of the comparative permanent magnet MAG0 of FIG. 5b.

The comparative magnet MAG0 of FIG. 5a or 5b may also form two distinct collecting regions. The comparative magnet MAG0 may also collect particles to two collecting regions. An upper annular collecting region may be located slightly above the bottom of the cylindrical portion of the magnet MAG0, and a lower annular collecting region may be located slightly below the bottom of the cylindrical portion of the magnet MAG0. A position H_{0L} may denote a vertical position of a lower annular collecting region of the comparative magnet MAG0, with respect to the bottom of the vessel. The relative difference ΔH₀₁/H_{0L} may depend on the shape of the convex bottom portion CNX1. The shape of the convex bottom portion CNX1 may be selected such that the relative difference ΔH₀₁/H_{0L} is, e.g., in the range of 10% to 60%. The shape of the convex bottom portion CNX1 may be selected such that the relative difference ΔH₀₁/H_{0L} is, e.g., in the range of 30% to 60%.

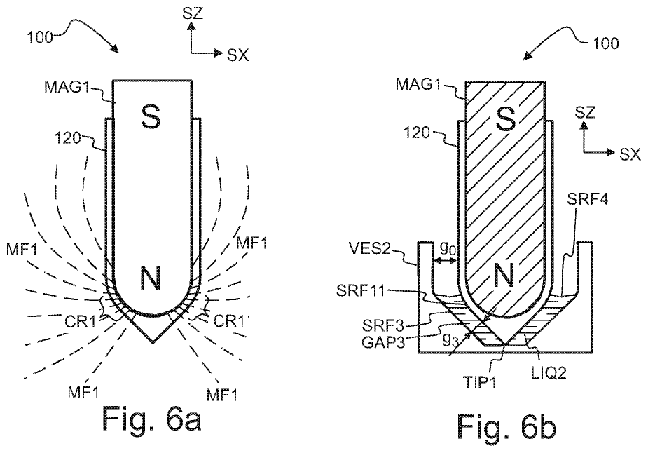

FIG. 6a shows, by way of example, a magnetic field MF1 generated by a probe magnet MAG1, which has a convex bottom portion CNX.

Referring to FIG. 6b, the shape of the vessel VES1 and/or VES2 may be optionally selected to correspond to the shape of the outer surface of the probe 100. The internal shape of the vessel may substantially correspond to the external shape of the shield 120 of the probe 100.

A tip TIP1 of the shield 120 may be optionally brought in contact with the bottom of the vessel VES1 or VES2 so that a gap GAP3 having a width g₃ remains between the shield 120 and the vessel. The gap GAP3 may also be called, e.g., as an interstice. The width g₃ of the wetted gap GAP3 between the shield 120 and the vessel VES1 and/or VES2 may be, e.g., in the range of 0.05 mm to 0.2 mm. The width g₃ of the gap GAP3 may be measured in a direction, which is perpendicular to the outer surface of the shield 120. Using a small gap width may allow reducing the minimum volume of the liquid LIQ1 or LIQ2. A non-zero width g₃ of the gap GAP3 may also reduce a risk of compressing the particles P1 between the collection region CR1 and the vessel VES1. Thus, the gap may reduce a risk of damaging particles P1 attached to the collection region CR1.

The shape of the vessel VES1 may be selected such that the gap is large at vertical positions above a nominal upper level SRF4 of the liquid, so as to ensure that only the bottom portion of the shield 120 is wetted during the operation. The width go of the gap may be, e.g., greater than 1.0 mm above the nominal upper level SRF4 of the liquid LIQ1.

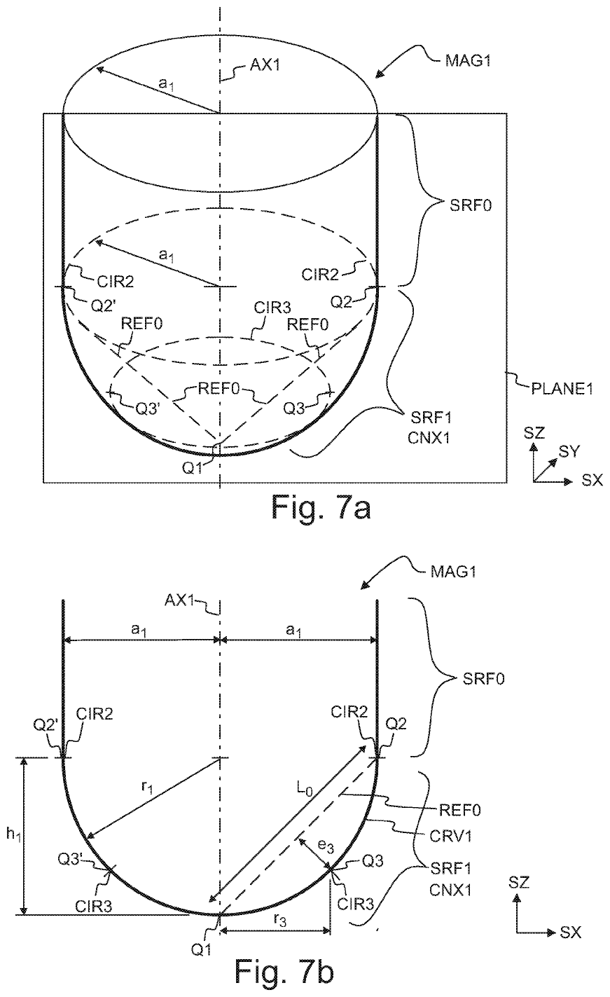

The geometry of the convex bottom portion CNX is now discussed with reference to FIGS. 7a and 7b. The cylindrical portion SRF0 of the magnet MAG1 may have a circular lower boundary CIR2. The symmetry axis AX1 of the magnet MAG1 may intersect the bottom portion CNX1 at a point Q1. The boundary CIR2 and the intersection point Q1 may define a conical reference surface REF0. The surface SRF1 of the convex bottom portion CNX1 of the magnet MAG1 may protrude by a distance e₃ with respect to the conical reference surface REF0.

The symbol a₁ may denote the radius of the cylindrical portion SRF0. The diameter D_{MAG1} of the magnet MAG1 may be equal to two times the radius a₁. The symbol a₁ may denote the height of the bottom portion CNX1 of the magnet MAG1. Lo may denote the slant length of the conical reference surface REF0. Lo may denote the distance between the intersection point Q1 and the circular boundary CIR2.

The surface SRF1 of the convex bottom portion CNX1 may have a circular protrusion region CIR3 which has the maximum protrusion distance e₃ with respect to the conical reference surface REF0. The distance e₃ may be, e.g., greater than or equal to 10% of the radius a₁ of the cylindrical portion. The circular region CIR3 may have a radius r₃. The radius r₃ may be, e.g., in the range of 10% to 90% of the radius a₁ of the cylindrical portion SRF0.

A vertical reference plane PLANE1 may contain the symmetry axis AX1 of the magnet MAG1. The symbol CRV1 may denote an intersection curve of the vertical reference plane PLANE1 and the surface SRF1 of the magnet MAG1. The vertical reference plane PLANE1 may intersect the boundary CIR2 at the points Q2 and Q2′. The vertical reference plane PLANE1 may intersect the circular region CIR3 at the points Q3 and Q3′. The protrusion distance e₃ may be equal to the distance of the point Q3 from the line defined by the points Q1 and Q2.

The intersection curve CRV1 may have a radius r₁. The bottom portion CNX1 may be, e.g., a hemispherical portion. In that case the radius r₁ may be equal to the radius a₁ when z<h₁.

The intersection curve CRV1 may have a radius r₁(z), which may depend on the vertical position z. For example, the surface SRF1 of the bottom portion CNX1 may be, e.g., a semi-ellipsoidal surface.

SX, SY and SZ denote orthogonal directions. The direction SZ may be substantially parallel with the symmetry axis AX1 of the magnet MAG1. The direction SZ may be a substantially vertical direction. The direction SZ may be substantially anti-parallel (i.e., opposite to) with the direction of gravity. A movement upwards may mean a movement in the direction SZ, and a movement downwards may mean a movement in the opposite direction −SZ.

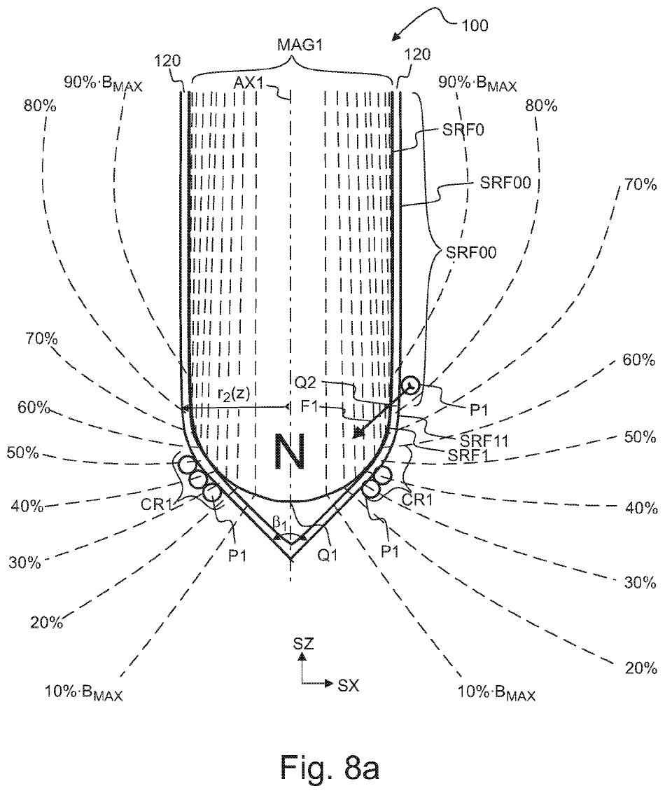

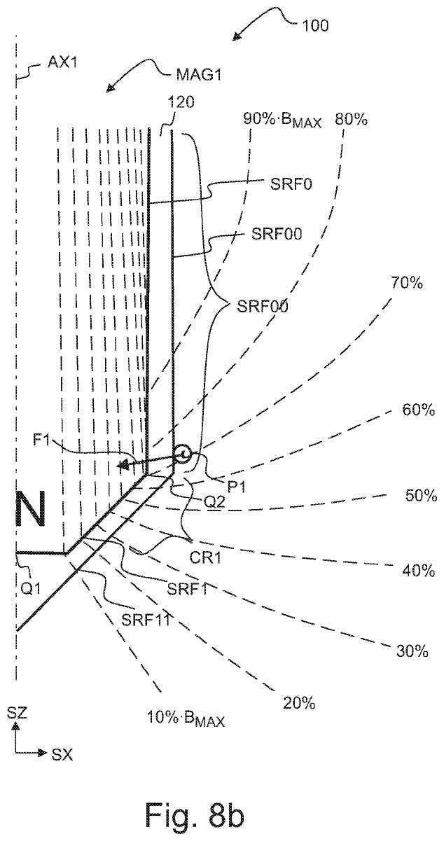

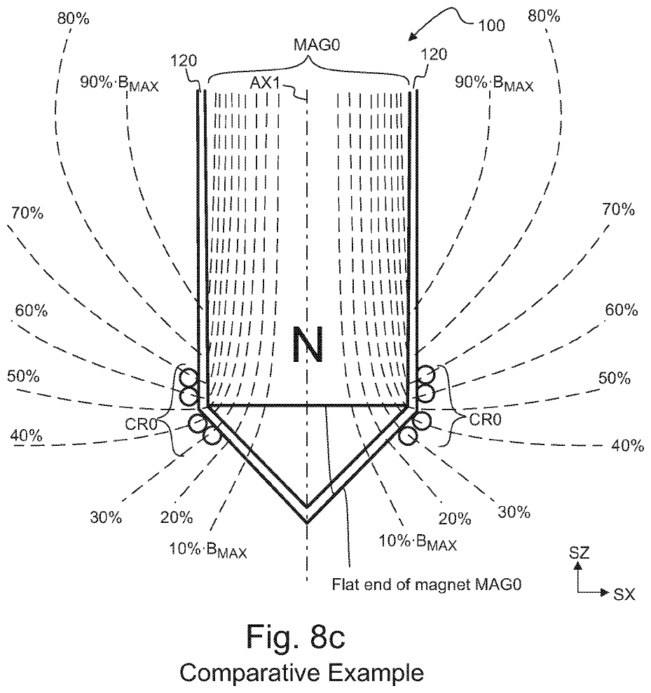

FIG. 8a shows, by way of example, the spatial distribution of the magnitude of the magnetic field MF1 generated by a permanent magnet MAG1, which has a hemispherical bottom portion CNX1. B_{MAX} denotes a maximum value of the magnetic field generated at the surface SRF00 of the shield 120 of the probe 100. SRF1 denotes the surface of the convex bottom portion of the magnet MAG1. SRF11 denotes the bottom surface portion of the shield 120. SRF0 denotes the cylindrical surface portion of the magnet MAG1. SRF00 denotes the cylindrical surface portion of the shield 120.

It may be noticed that the maximum gradient of the magnetic field is located below the point Q2, i.e., below the boundary CIR2 of the cylindrical portion SRF0.

The doubly curved shape of the convex portion CNX1 may provide the collection region CR1 where the magnetic field has maximum gradient. The curve CRV1 which defines the shape of the axially symmetric convex portion CNX1 may be curved have a finite radius of curvature in the vicinity of the collection region CR1. In other words, the curve CRV1 may be curved in the vicinity of the collection region CR1. The doubly curved convex portion CNX1 may guide and produce magnetic field such that the magnitude of the magnetic field may have large gradient at the collection region CR1.

Most of the particles P1 may be attached to the collection region CR1, which substantially coincides with the maximum gradient of the magnetic field, on the outer surface of the shield 120.

Interaction with the magnetic field may generate a pulling force F1, which may pull the particle P1 towards the shield 120 of the probe 100.

A moving particle P1 may sometimes impinge also on a cylindrical portion SRF00 of the shield above the boundary (Q2, CIR2). A transverse component of the magnetic force F1 may subsequently move the particle P1 downwards from the cylindrical portion SRF00 to the collecting region CR1 located at the bottom surface portion SRF11.

The cylindrical portion SRF0 of the magnet MAG1 may smoothly join the bottom portion SRF1 of the magnet MAG1 so as to facilitate movement of the particle P1 from the cylindrical portion SRF00 to the bottom surface portion SRF11. The cylindrical portion SRF0 may smoothly join the bottom portion SRF1 without a shoulder between the portions SRF0, SRF1. The cylindrical portion SRF0 may smoothly join the bottom portion SRF1 without an edge between the portions SRF0, SRF1. The radius of curvature of the intersection curve CRV1 may be, e.g., greater than 10% of the radius a₁ of the magnet MAG1 at all vertical positions z of the curve CRV1 in the range of 50% h₁ to 150% h₁. The point Q1 is located at the vertical position z=0.

In an embodiment, the cylindrical portion SRF00 of the shield 120 may optionally smoothly join the bottom portion SRF11 of the shield 120, without an edge, so as to facilitate movement of the particle P1 from the cylindrical portion SRF00 to the bottom surface portion SRF11. The minimum radius r₂(z) of curvature of the surface (SRF11, SRF00) of the shield (120) may be, e.g., greater than 10% of the radius (a₁) of the probe magnet (MAG1) at vertical positions (z) which are in the range of 50% to 150% of the height (h₁) of the convex bottom portion (CNX1). The radius r₂(z) of curvature may mean the radius r₂(z) of curvature of the outer surface of the shield 120 in the vertical plane (PLANE1). In case of a hemispherical bottom portion CNX1, the minimum radius r₂(z) may be, e.g., substantially equal to the 50% of the outer diameter D₁₂₀ of the shield 120.

Referring to FIG. 8b, an edge at the boundary CIR2 of the cylindrical portion SRF0 and the bottom portion SRF1 may have an effect on the direction of the magnetic force F1. The magnetic force F1 may be almost perpendicular to the surface SRF00 in the vicinity of the edge. The transverse vertical component of the magnetic force F1 near the edge shown in FIG. 6b may be weaker than in the situation of FIG. 6a where the bottom portion SRF1 smoothly joins the cylindrical portion SRF0. Furthermore, an edge of the shield 120 between the surface portions SRF00, SRF11 of the shield 120 may prevent movement of a particle P1 from the portion SRF00 to the portion SRF11.

FIG. 8c is a comparative example, which shows the spatial distribution of the magnitude of the magnetic field MF1 generated by a reference (permanent) magnet MAG0, which has a flat bottom end. The maximum gradient of the reference magnet MAG0 may be located at higher position, when compared, e.g., with the probes shown in FIGS. 8a and 8b. A collecting region CR0 provided by using the reference magnet MAG0 may be at higher position, when compared, e.g., with the collection regions CR1 of the probes shown in FIGS. 8a and 8b.

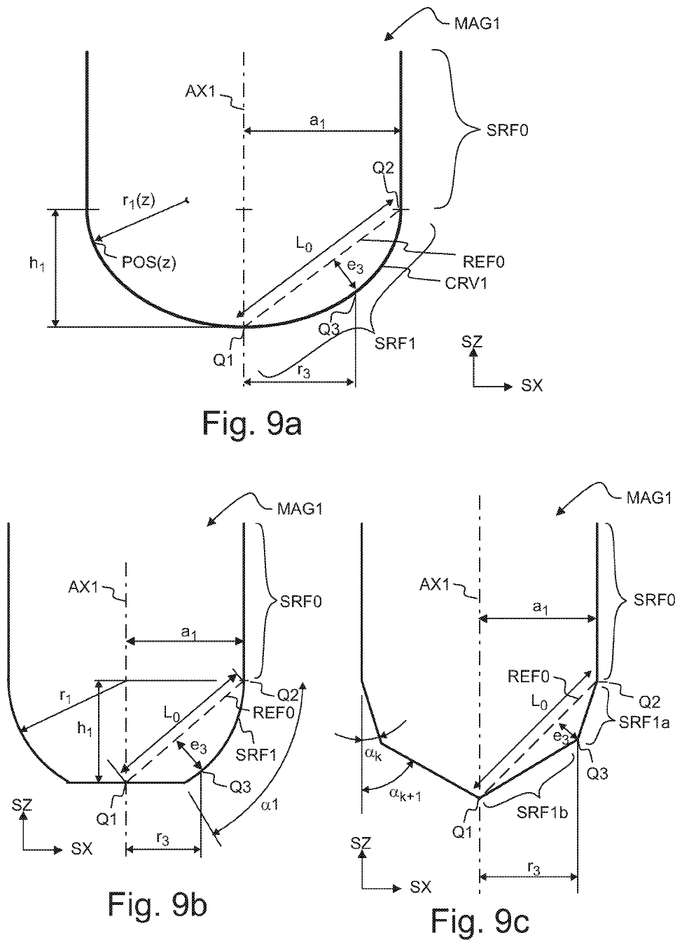

Referring to FIG. 9a, the radius r₁(z) of curvature of the surface of the convex bottom portion CNX1 of the magnet MAG1 at a may depend as a function of the vertical position z. The boundary CIR2 of the cylindrical portion SRF0 is at the vertical position POS(z=h₁). The boundary CIR2 intersects the curve CRV1 at the point Q2.

The surface SRF1 of the bottom portion CNX1 may be, e.g., a semi-ellipsoidal surface.

The height h₁ of the bottom portion CNX1 may be smaller than the radius a₁ of the cylindrical portion SRF0. The surface SRF1 of the bottom portion CNX1 may be, e.g., a portion of an oblate spheroid surface.

The height h₁ of the bottom portion CNX1 may be greater than the radius a₁ of the cylindrical portion SRF0. The surface SRF1 of the bottom portion CNX1 may be, e.g., a portion of a prolate spheroid surface.

Referring to FIG. 9b, the surface SRF1 of the bottom portion CNX1 may be a truncated hemispherical surface. α1 may denote the angular dimension (angular height) of the spherical portion of the bottom portion CNX1. In case of a truncated hemispherical surface, the height h₁ may be, e.g., greater than or equal to 30% of the radius a₁ (and smaller than 100% of the radius a₁).

Referring to FIG. 9c, the surface SRF1 of the bottom portion CNX1 may be a combination of conical surfaces SRF1a, SRF1b. αₖ may denote the cone angle of the first conical surface SRF1a. αₖ₊₁ may denote the cone angle of the second conical surface SRF1b. The cone angle (αₖ₊₁, αₖ) may decrease with increasing vertical coordinate z, so as to provide the convex shape.

The surface SRF1 of the bottom portion CNX1 may also be, e.g., a combination of a spherical surface and a conical surface. The surface SRF1 of the bottom portion CNX1 may also be, e.g., a truncated conical surface.

The surface SRF1 of the bottom portion CNX1 may be, e.g., a hemispherical surface, a truncated hemispherical surface, a truncated conical surface, a combination of conical surface portions, a semi-ellipsoid surface, a prolate semi-ellipsoid surface, an oblate semi-ellipsoid surface, a truncated semi-ellipsoid surface, a paraboloid surface, a truncated paraboloid surface.

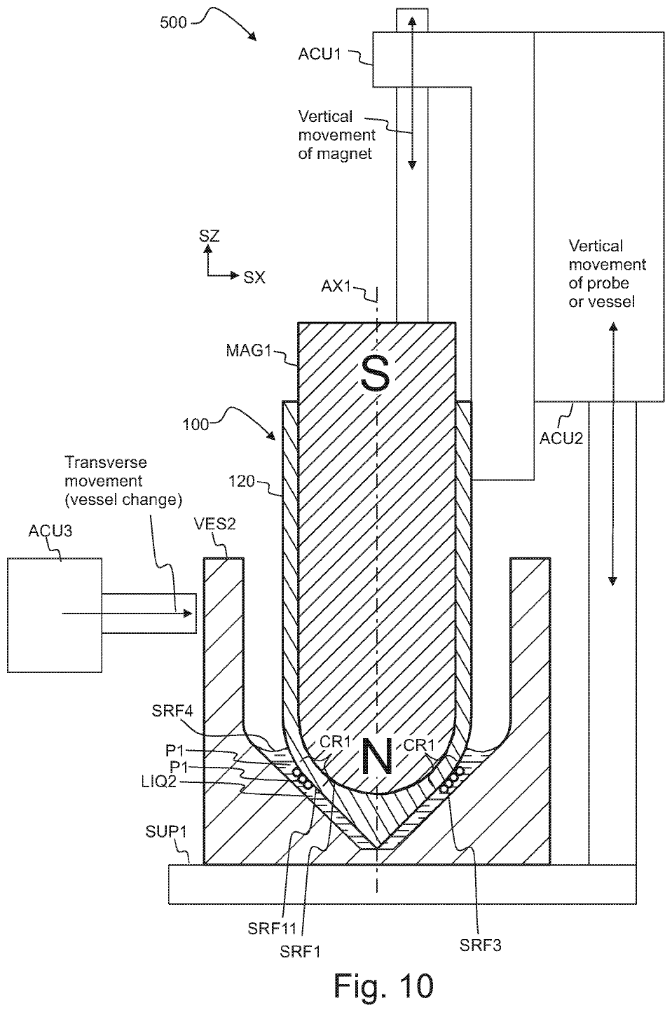

Referring to FIG. 10, an apparatus 500 may comprise:
a support (SUP1) for holding a vessel (VES1) for containing a composition (MX1), which comprises a target substance (M1), a first liquid (LIQ1) and magnetically responsive particles (P1),
a transfer probe (100), which comprises a shield (120) and a probe magnet (MAG1) movable inside the shield (120),
a first actuator (ACU1) for causing relative movement of the probe magnet (MAG1) with respect to the shield (120),
a second actuator (ACU2) for causing relative movement of the shield (120) with respect to the vessel (VES1), wherein the apparatus (500) is arranged:
to collect the magnetically responsive particles (P1) to the transfer probe (100) by introducing a bottom end of the transfer probe (100) into the vessel (VES1),
to lift the magnetically responsive particles (P1) together with the transfer probe (100) from the vessel (VES1) by moving the transfer probe (100) and/or by moving the vessel (VES1),
to position the transfer probe (100) to a release location (LOC2,VES2), and
to release the magnetically responsive particles (P1) from the probe (100) to the release location (LOC2,VES2) by moving the probe magnet (MAG1) with respect to the shield (120),

The vessel VES1 or VES2 may have an inner surface SRF3. The liquid LIQ1 or the sample MX0, MX1, MX2 may have an upper surface SRF4.

The collected particles may be optionally analyzed. The collected particles may be subsequently analyzed, e.g., by using an analytical instrument. The method may comprise, e.g., detecting and/or measuring the target substance M1 transferred by using the magnetic probe 100. The method may comprise, e.g., measuring an amount or a concentration of the target substance M1 transferred by using the magnetic probe 100. The method may comprise, e.g., detecting and/or measuring magnetic particles P1 transferred by using the magnetic probe 100. The method may comprise, e.g., detecting and/or measuring a parameter related to the target substance M1 transferred by using the magnetic probe 100. The method may comprise, e.g., determining whether a sample MX0 comprises the target substance M1 or not.

The apparatus 500 may be arranged to collect the target substance M1 from a mixture MX1 in order to produce a product. The apparatus 500 may be arranged to increase the concentration of the target substance M1 in order to produce a product. The apparatus 500 may be arranged to process the target substance M1 in order to produce a product. The product may be, e.g., a medicament.

The volume of the liquid LIQ2 may be substantially smaller than the volume of the liquid LIQ1 of the original sample MX0. The method may comprise increasing the concentration of the target substance M1, by collecting the particles P1 from the sample MX0 and by transferring the collected particles P1 to a release location LOC2. An enriching ratio of the method may mean the ratio of the concentration of the target substance M1 in the second liquid LIQ2 of the release location LOC2 to the concentration of the target substance M1 in the first liquid LIQ1 of the composition MX1 of the first vessel VES1. The enriching ratio may be, e.g., greater than 2, greater than 10, or even greater than 100.

The apparatus 500 may be arranged to separate cells. The apparatus 500 may be arranged to separate biomolecules. The apparatus 500 may be arranged to enrich biomolecules.

The second actuator ACU2 may be arranged to cause a relative movement between the probe 100 and the vessel VES1 and/or VES2. For example, the actuator ACU2 may move the probe 100 with respect to the vessel and/or the actuator ACU2 may move the vessel with respect to the probe 100.

The actuator ACU2 may be arranged to cause a relative movement between the shield 120 and the vessel VES1 and/or VES2. For example, the actuator ACU2 may move the shield 120 with respect to the vessel and/or the actuator ACU2 may move the vessel with respect to the shield 120.

For example, the actuator ACU2 may be arranged to bring the bottom of the vessel VES1 and/or VES2 in contact with the bottom portion of the shield 120.

For example, the second actuator ACU2 may be arranged to bring the bottom of the vessel VES1 and/or VES2 close to the shield 120.

The apparatus 500 may be optionally arranged to cause the relative movement between the probe and the vessel such that a gap width g₃ between the shield 120 and the vessel is kept greater than a predetermined limit value, in order to minimize or prevent crushing the particles.

The apparatus 500 may optionally comprise, e.g., a resilient element in order to allow pushing the shield 120 in contact with the vessel VES1 and/or VES2, without damaging one or more parts of the apparatus. The apparatus 500 may optionally comprise, e.g., a force sensor and a control system, which may be arranged to keep an actuating force of the second actuator ACU2 below a predetermined limit, in order to allow pushing the shield 120 in contact with the vessel, without damaging one or more parts of the apparatus.

Release of the particles P1 may be optionally facilitated, e.g., by vibrating the probe 100. The particles P1 may be released from the probe 100 to the release location LOC2, e.g., by vibrating of the shield. The apparatus may comprise, e.g., a vibrating transducer to cause temporary vibration of the shield.

The apparatus 500 may optionally comprise an actuator ACU2, ACU3 for moving the probe 100 from a first vessel VES1 to a second vessel VES2. The apparatus 500 may optionally comprise an actuator ACU2, ACU3 for causing relative movement of the probe 100 with respect to a first vessel VES1 and for causing relative movement of the probe 100 with respect to a second vessel VES2. For example, an actuator ACU2, ACU3 may move the probe 100 in a transverse direction with respect to the vessels VES1, VES2. For example, an actuator ACU2, ACU3 may move the vessel VES1 and/or VES2 in a transverse direction with respect to the probe 100. The actuator ACU2, ACU3 may comprise, e.g., a rotating support for causing a relative transverse movement of the vessels VES1, VES2 with respect to the probe 100.

The apparatus 500 may comprise a support SUP1 for holding one or more vessels (VES1, VES2). The support SUP1 may be arranged to hold, e.g., a well plate, which comprises an array of wells. The support SUP1 may be, e.g., a tray for holding a well plate. An actuator (e.g., ACU2 and/or ACU3) may be arranged to cause relative movement between the probe 100 and a vessel (VES1, VES2) by causing relative movement between the probe 100 and the support 100. The support SUP1 may be stationary, or an actuator (e.g., ACU2 and/or ACU3) may be arranged to move the support SUP1, e.g., in a vertical direction. The apparatus 500 may further comprise the one or more vessels (VES1, VES2). The vessels (VES1, VES2) may be consumable and/or replaceable parts. The vessel (VES1, VES2) may be replaced, e.g., in order to ensure that the inner surface is clean.

Referring to FIGS. 11a to 11d, the apparatus may optionally comprise one or more auxiliary magnets MAG2 to facilitate releasing the particles P1 from the probe 100 to the release location LOC2. The particles P1 may be pulled from the probe 100 to the release location LOC2 by magnetic forces caused by an auxiliary magnet MAG2, in a situation where the magnetic field of the probe 100 is temporarily reduced.

The particles P1 may be attracted from the shield 120 towards the release location LOC2 by means of one or more auxiliary release magnets MAG2 placed under the release location LOC2. The auxiliary magnet MAG2 may be a permanent magnet or an electromagnet.

The probe 100 may be moved and contacted with a release surface and/or vessel at the release location LOC2. The probe magnet MAG1 may be moved upwards, whereby by the release magnet MAG2 may attract the particles P1 to form a concentrated spot on the release location LOC2.

The convex bottom portion CNX of the magnet MAG1 may facilitate releasing the particles P1 to a thin layer of a liquid film LIQ2.

The release location LOC2 may also be implemented, e.g., by using a plate PLA2. The collected particles P1 may be released to a release surface SRF2. The collected particles P1 may be released, e.g., to a release surface SRF2 of a plate PLA2. The plate PLA2 may be, e.g., a microscope slide or a growing substrate. The plate PLA2 may be, e.g., a glass plate. A portion of a growing substrate may be used as the release location LOC2. The growing substrate may be, e.g., a petri dish. The growing substrate may be, e.g., an agar substrate. The method may be used, e.g., in order to study growth of fungi or bacteria.

The apparatus 500 may be arranged to carry out the method automatically. The method may also be applied as a manual method, or as a semi-automatic method.

The probe magnet MAG1 may comprise, e.g., rare earth magnet material. The probe magnet MAG1 may comprise, e.g., neodymium magnet alloy or samarium-cobalt magnet alloy.

Using a permanent magnet to generate the collecting magnetic field may provide one or more of the following technical effects, when compared with an electromagnet:
smaller size, as the electromagnetic coil is not needed,
high and stable magnetic field,
reduced consumption of energy,
no heating due to electric current of the coil of the electromagnet,
emission of electromagnetic radiation from the coil may be avoided.

The sheath 120 may optionally have a substantially constant thickness. The bottom of the shield 120 of the transfer probe 100 may have, e.g., a substantially constant thickness, e.g., in order to facilitate producing the shield 120 and/or in order to reduce an amount of material needed for producing the shield 120.

Referring to FIGS. 12a and 12b, the shield 120 of the transfer probe 100 may have, e.g., a spherical outer surface SRF11. A collecting region CR1 provided by the spherical bottom surface SRF11 may also comprise a central portion of the surface SRF11. Some particles P1 may be attracted also to locations of the surface SRF11, which are close to the axis AX1 of the magnet MAG1. However, the transfer probe 100 with the spherical outer surface SRF11 may also allow collecting the particles P1 from a small volume and/or may allow releasing the particles P1 to a small volume.

The bottom surface SRF3 of the vessel VES1, and/or VES2 may have, e.g., a tapered shape. The bottom surface SRF3 of the vessel VES1, and/or VES2 may have a tapered shape, e.g., in order to reduce an amount of the liquid LIQ1, LIQ2 needed for collecting and/or releasing the particles P1 with the probe 100. The bottom surface SRF3 of the vessel VES1, and/or VES2 may have a tapered shape, e.g., in order to funnel the liquid LIQ1, LIQ2 to a central portion of the vessel VES1, and/or VES2.

FIG. 12c shows, by way of example, the shape of the bottom of the vessel shown in FIGS. 12a, 12b, 12f. The tapered bottom surface SRF3 of FIG. 12d may funnel the liquid LIQ1, LIQ2 to a central recessed portion REC1 of the vessel. The bottom surface SRF3 may comprise a recess REC1. The taper angle of the bottom surface SRF3 of the vessel may depend on the radial position, so as to provide a recessed portion REC1. The tapered bottom surface SRF3 of FIG. 12d may allow operation with a small amount of the liquid LIQ1, LIQ2. The tapered bottom surface SRF3 of the vessel may have a first taper angle γ31 at a first radial position r₃i, and a second different taper angle γ32 at a second radial position r₃₂. The first taper angle γ₃₁ may be, e.g., in the range of 40° to 60°, and the second taper angle γ₃₂ may be, e.g., in the range of (γ₁₁+1°) to (γ₁₁+20°). The first taper angle γ₃₁ may be, e.g., in the range of 50° to 55°, and the second taper angle γ₃₂ may be, e.g., in the range of)(γ31+5°) to (γ₃₁+10°). The first radial position r₃₁ may be, e.g., at 25% of the radius α1 of the magnet MAG1. The second radial position r₃₂ may be, e.g., at 50% of the radius a₁ of the magnet MAG1. The bottom of the vessel may have a symmetry axis AX0. The radial positions r₃₁, r₃₂ may be defined with respect to the axis AX0.

Referring to FIG. 12d, a transfer probe 100 with a tip TIP1 may also be used together with the vessel of FIG. 12a. The tip TIP1 of the shield 120 may, e.g., facilitate collecting particles P1 from a composition MX1. The tip TIP1 of the shield 120 may, e.g., facilitate releasing collected particles P1 to a liquid LIQ2. The tip may facilitate collecting, e.g., when the composition MX1 has high viscosity. The tip may facilitate release, e.g., when the liquid LIQ2 has high viscosity. The tip TIP1 may, e.g., cause a stirring effect in the composition MX1 and/or in the liquid LIQ2. The tip TIP1 may also reduce a risk of damaging the particles P1. The tip TIP1 may optionally ensure that a gap GAP3 may remain between the collecting region CR1 and the bottom surface SRF3 of the vessel.

Referring to FIGS. 12e and 12f, the outer diameter D₁₂₀ of the shield 120 may be, e.g., in the range of 105% to 200% of the diameter D_{MAG1} of the magnet MAG1. The diameter D_{MAG1} of the magnet MAG1 may be substantially smaller than the diameter D₁₂₀ of the shield 120, e.g., in order to ensure that the particles P1 are attracted to a bottom portion 125 of the shield 120 and/or in order to further reduce the minimum amount of liquid (LIQ1, LIQ2) needed for transferring the particles P1 with the probe 100. The outer diameter D₁₂₀ of the shield 120 may be, e.g., in the range of 120% to 200% of the diameter D_{MAG1} of the magnet MAG1.

The shield 120 may comprise a bottom portion 125. The shield 120 may comprise a tapered bottom portion 125. The shield 120 may comprise a tapered bottom portion 125 with a tip TIP1. The shield 120 may optionally comprise a centering portion 128 to define a transverse position of the shield 120 with respect to the magnet MAG1. The outer diameter D128 of the centering portion 128 may be smaller than or equal to the outer diameter D₁₂₀ of the shield 120. The outer diameter D128 of the centering portion 128 may be substantially smaller than the outer diameter D₁₂₀ of the shield 120. The shield 120 may optionally comprise, e.g., an annular protrusion 127 between the bottom portion 125 and the centering portion 128.

Referring to FIG. 12g, the apparatus 500 may comprise an array of transfer probes 100a, 100b, 100c, 100d. Each probe may comprise a magnet (MAG1a, MAG1b, MAG1c, MAG1d), and a shield portion (120a, 120b, 120c, 120d). The magnets may be connected to a common support 150. The shield portions may be connected to each other, e.g., by joining portions 122. The shield portions (120a, 120b, 120c, 120d) and the joining portions 122 may together form an array of shields. The shield array may also be called, e.g., as a comb. The apparatus 500 may comprise an array of vessel portions VES1a, VES1b, VES1c, VES1d. The vessel portions may also be called as wells. The wells VES1a, VES1b, VES1c, VES1d may together constitute, e.g., a well plate. Each vessel portion may contain a composition (MX1). Each vessel portion may contain a different composition (MX1). The apparatus may be arranged to simultaneously move the transfer probes 100a, 100b, 100c, 100d with respect to the wells and/or the apparatus may be arranged to simultaneously move the wells VES1a, VES1b, VES1c, VES1d with respect to the transfer probes. The apparatus may be arranged to simultaneously process a plurality of compositions (MX1) contained in the wells VES1a, VES1b, VES1c, VES1d. The shapes of the magnets, the shield portions and/or the wells may be selected, e.g., as disclosed above with reference to FIGS. 2 to 12f.

Referring to FIG. 12h, the apparatus 500 may comprise an array of transfer probes 100a, 100b, 100c, 100d. Each probe may comprise a magnet (MAG1a, MAG1b, MAG1c, MAG1d), and a sheath portion (120a, 120b, 120c, 120d). Each magnet (MAG1a, MAG1b, MAG1c, MAG1d) may have a convex bottom portion (CNX1). The magnets may be connected to a common support 150. The magnets may be oriented so that their N and S poles are inverted. For example, the orientation the poles (N,S) of a second probe magnet MAG1b may be inverted with respect to orientation the poles (S,N) of a first probe magnet MAG1a, in a situation where the second probe magnet MAG1b is adjacent to the first probe magnet MAG1a. For example, the magnetic dipole moment of the first probe magnet (e.g., MAG1a) of the array may have a first direction (e.g., downwards), and the magnetic dipole moment of at least a second probe magnet (e.g., MAG1b) of the array may have a second opposite direction (e.g., upwards). The magnets may be oriented so that the orientation of at least one magnet is inverted. This may reduce the combined magnetic far field surrounding the array of the magnets and/or may equalize and increase the combined magnetic near field between the bottom ends of adjacent magnets (MAG1a, MAG1b). This may equalize and/or increase the particle collecting efficiency of the adjacent probes 100. Thus, a smaller (average) amount of liquid may be used for releasing the particles. The apparatus 500 of FIG. 12h may otherwise correspond to the apparatus 500 of FIG. 12g, wherein at least one magnet of the array may have inverted magnetic orientation with respect to at least one second magnet of the array.

For the person skilled in the art, it will be clear that modifications and variations of the devices and the methods according to the present invention are perceivable. The figures are schematic. The particular embodiments described above with reference to the accompanying drawings are illustrative only and not meant to limit the scope of the invention, which is defined by the appended claims.

1. A method for processing a composition by using a magnetic transfer probe, the transfer probe comprising a shield and a probe magnet movable inside the shield,
the method comprising:
providing a first composition in a vessel, wherein the composition comprises a first liquid and a plurality of magnetically responsive particles, wherein the particles are arranged to selectively interact with a target substance,
positioning the transfer probe into the vessel so as to collect the particles from the first composition,
removing the collected particles together with the transfer probe from the vessel by causing a relative vertical movement between the transfer probe and the vessel, and
releasing the collected particles from the shield to a release location by causing a relative vertical movement between the probe magnet and the shield,
wherein the probe magnet is a permanent magnet, which comprises a cylindrical portion and a convex bottom portion adjoining the cylindrical portion, the magnet has an axis of symmetry, the axis of symmetry intersects the bottom portion at an intersection point, the intersection point and the circular lower boundary of the cylindrical portion define a reference cone, and the bottom portion protrudes with respect to the reference cone.

2. The method of claim 1, wherein the surface of the convex bottom portion has maximum distance from the reference cone at a circular region, wherein said maximum distance is greater than or equal to 10% of the radius of the cylindrical portion.

3. The method of claim 1, wherein the surface of the convex bottom portion has maximum distance from the reference cone at a circular region, wherein the radius of said circular region is in the range of 10% to 90% of the radius of the cylindrical portion.

4. The method according to claim 1, wherein the surface of the convex bottom portion has maximum distance from the reference cone at a circular region, wherein the vertical position of said circular region is in the range of 10% to 90% of the height of the convex bottom portion.

5. The method according to claim 1, wherein the ratio of the height of the convex bottom portion to the radius of the cylindrical portion is in the range of 0.5 to 2.0, advantageously in the range of 0.7 to 1.5.

6. The method according to claim 1, wherein the convex bottom portion is a hemisphere or a truncated hemisphere.

7. The method according to claim 1, wherein the convex bottom portion is a half spheroid, a truncated half spheroid, a truncated cone, or a combination of two or more conical portions with different taper angles.

8. The method according to claim 1, wherein the diameter of the probe magnet in the range of 1 mm to 8 mm.

9. The method according to claim 1, comprising releasing the collected particles from the shield to a liquid in a second vessel, wherein the volume of the liquid in the second vessel is in the range of 5 μl to 50 μl, advantageously in the range of 5 μl to 15 μl.

10. The method according to claim 1, comprising releasing the collected particles from the shield to a surface.

11. The method according to claim 1, wherein the particles are ferromagnetic particles, ferrimagnetic particles, or superparamagnetic particles, and wherein the size of the particles is in the range of 50 nm to 10 μm.

12. An apparatus, comprising:
a support for holding a vessel for containing a composition, which comprises a first liquid and magnetically responsive particles,
a transfer probe, which comprises a shield and a probe magnet movable inside the shield,
a first actuator for causing relative vertical movement between the probe magnet and the shield,
a second actuator for causing relative vertical movement between the transfer probe and the vessel,
wherein the apparatus is arranged:
to position the transfer probe into the vessel so as to collect the particles from the first composition,
to remove the collected particles together with the transfer probe from the vessel by moving the transfer probe upwards and/or by moving the vessel downwards, and
to release the collected particles from the shield to a release location by moving the probe magnet upwards with respect to the shield and/or by moving the shield downwards with respect to the probe magnet,
wherein the probe magnet is a permanent magnet, which comprises a cylindrical portion and a convex bottom portion adjoining the cylindrical portion, the magnet has an axis of symmetry, the axis of symmetry intersects the bottom portion at an intersection point, the intersection point and the circular lower boundary of the cylindrical portion define a reference cone, and the bottom portion protrudes with respect to the reference cone.

13. The apparatus of claim 12, wherein the convex bottom portion is a hemisphere or a truncated hemisphere.

14. The apparatus of claim 12 or 13, wherein the diameter of the probe magnet is in the range of in the range of 1 mm to 8 mm.

15. The apparatus according to claim 12, comprising an array of probe magnets, wherein at least one magnet of the array has an inverted magnetic orientation with respect to at least one second magnet of the array.
